(19) United States
(12) Patent Application Publication
(10) Pub. No.: US 2020/0251193 A1
(43) Pub. Date: Aug. 6, 2020

(54) SYSTEM AND METHOD FOR INTEGRATING GENOTYPIC INFORMATION AND PHENOTYPIC MEASUREMENTS FOR PRECISION HEALTH ASSESSMENTS

(71) Applicant: Multimodal Imaging Services Corporation, San Diego, CA (US)

(72) Inventors: Nathan S. White, San Diego, CA (US); Chun C. Fan, San Diego, CA (US); Anders M. Dale, San Diego, CA (US)

(21) Appl. No.: 15/985,386

(22) Filed: May 21, 2018

(51) Int. Cl. G16H 10/60; G16H 50/30; G16H 50/20; G16B 20/20

(52) U.S. Cl. CPC G16H 10/60; G16B 20/20; G16H 50/20; G16H 50/30

(57) ABSTRACT

The present disclosure is directed to a system and method to integrate genotypic information and phenotypic measurements for predicting health related risks. While the genetic information is extracted through efficient training with genotypic data and biological priors, the phenotypic measurements are further integrated into the risk assessing model through updating. The flexibility of this approach enables not just personalized risk assessment in near future, but also a framework to evaluate the value of specific medical tests, clinical decision support, and life actuarial calculations.

### BACKGROUND

This present disclosure is directed to bioinformatics and statistical inference, focusing on health-related risk prediction. The system and method integrate phenotypic measurement data associated with an individual with the individual's germline genetic information. The phenotype measurement data may include, but is not limited to, biomedical or health care records, bioassays, medical imaging data, cognitive performance data and/or neuropsychological test data, behavioral assessments, blood and/or metabolic test data, physiologic data, and the like, and combinations thereof. The integration approach may provide short term/long term health prediction, evaluation of specific tests, clinical or medical decision support, and life actuarial calculation.

The genotyping technology and large-scale genome-wide association studies (GWAS) have enabled disease risk prediction based on genetic information. This drives a surge of hope that personalized risk assessment may be achieved through genetic risk predictions. Current practice for generating genetic risk prediction involves training a model based on existing GWAS and then applying the learnt model to individuals who were not part of the training cohort. Until 2017, the most popular method for genetic risk prediction involved using several genetic markers (single nucleotide polymorphisms, SNPs) to generate a polygenic score, which is the weighted sum of an individual's genotypes:

$$S c o r {e}_{j} = {\sum }_{m} {X}_{j m} {β}_{m}$$

$${β}_{m} = log ( Odds Ratio )$$

Despite its popularity, an individual's polygenetic risk score is limited by the fact that the polygenic score captures a tiny fraction of heritability, a common statistic used to describe the degree of variation in a phenotypic trait in a population that is due to the genetic variations between individuals in that population. Therefore, roughly one third of the observed variations in a given trait or disease cannot be explained with polygenic scoring alone, even with a perfect polygenic test. This heritability constraint imposes an upper bound on the accuracy and prediction power of SNP-based risk tests for disease prediction. Only very recently did researchers begin to incorporate the genetic risk prediction into the risk calculations with other lifestyle risk factors.

Although, the research field gradually realized the importance of taking all risk factors into consideration, none have explicitly integrated baseline genetic information with phenotypic measurement. The phenotypic measurements are always treated as one of the outcomes as associations with genotypes are the main focus of the research. As discussed above, the genetic risk prediction needs additional context information in order to achieve personalized health assessment.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

The system and method of the present disclosure use a two-pronged approach to achieve a personalized health assessment. First, the genetic risk prediction is enhanced by provide a generic risk score for an individual. Second, a framework is then used to integrate the genetic risk score and phenotypic measurements through active updating. By integrating genotypes (the genetic construction of an individual) and phenotypes (a set of observations made on the individual, for example tests performed on the individual), the impact of measurement errors may be reduced and the prediction performance of a health or disease risk assessment may be improved beyond the heritability constraint of a given trait.

The system and method enable the integration of genotypic information and phenotypic measurements for personalized health assessment and lifetime disease risk prediction. The algorithm models the age-dependent process of disease/traits, outcome health risk prediction that is time dependent. The genotypic information is based on genetic risk prediction for non-age dependent risk tests. Efficient training of the genotype risk model, by incorporating biological priors and characteristics of training cohorts, allows for an improvement on the genetic risk prediction compared with traditional genotype risk models. The predicted genetic risks are then regarded as the baseline risk for the individual, while any additional phenotypic measurements are combined with the baseline risk to provide an updated risk prediction for the individual. The phenotypic measurements may further be augmented by comparing the measurement with reference standards (normative data) derived from, for example, phenotypic measurements from a similar cohort of individuals based on demographic information (e.g., age and sex) and/or genetic information (genetic informed norms). Germline genetic variants are those inherited by an individual and maintained invariant across the individual's life-span. These germline genetic variants may be regarded as a risk background the individual inherited. Any additional tests performed on the individual may be regarded as taking an observation or snapshot of the current status or health of the individual, which provides information on the physiological condition of the individual with test specific measurement errors. The predicted genetic risks and phenotypic measurements may then be integrated or combined to provide a personalized health assessment for that individual using, for example, the Bayes rule. The personalized health assessment may be used for lifetime disease risk prediction, evaluating the value of a specific test, and supporting the clinical decisions concerning the health of the individual.

In accordance with one embodiment of the present disclosure, there is provided a method for deriving a personalized health assessment for an individual by integrating selected genotypic information with phenotypic measurements associated with the individual, via a computing system. The computing system may comprise a processor operable to control the computing system, data storage operatively coupled to the processor, wherein data storage is configured to store a plurality of genotypic information, a plurality of phenotypic measurements, and combinations thereof, and an input/output device operatively coupled to the processor, wherein the input/output device is configured to receive a plurality of data for transmission to the processor, wherein the input/output device is configured to transmit a plurality of data generated by the processor. The computing system may also comprise a genetic risk prediction component operatively connected to the processor and controlled in part by the processor, wherein the genetic risk prediction component is configured to generate a plurality of genetically defined lifetime risks of having a plurality of diseases, and an integration component operatively coupled to the processor and controlled in part by the processor, wherein the integration component is configured to integrate genotypic information with phenotypic measurements.

In one embodiment, the method for deriving the personalized health assessment comprises receiving, via the input/output device, a plurality of trained genetic risk weights associated with a selected medical condition and transmitting the received trained genetic risk weights to the genetic risk prediction component. In one embodiment, the plurality of trained genetic risk weights comprises genetic data selected from the group consisting of genomic data, genotyped calls, imputed genetic data, sequence data, structural variations, copy number variations, and combinations thereof.

The method may further comprise receiving, via the input/output device, a plurality of germline genetic information associated with the individual and transmitting the received germline genetic information to the genetic risk prediction component. In a preferred embodiment, the plurality of germline genetic information comprises data selected from the group consisting of genotype data, genotyped calls, imputed genetic data, sequence data, structural variation data, copy number variations, and combinations thereof.

The method may also comprise subjecting, via the genetic risk prediction component, at least a portion of the received germline genetic information to a genetic risk prediction function using at least a portion of the plurality of trained genetic risk weights to generate at least one age-dependent genetic risk score for the individual.

In one embodiment, a plurality of phenotypic measurements associated with the individual is received via the input/output device and transmitted to the integration component. In a preferred embodiment, the plurality of phenotypic measurement data comprises data selected from the group consisting of biomedical record data, or health care record data, bioassay data, medical imaging data, cognitive performance data, neuropsychological test data, behavioral assessment data, blood analysis data, metabolic test data, physiologic data, and combinations thereof.

In one embodiment, at least a portion of the received phenotypic measurements is selectively integrated into the at least one age-dependent genetic risk score by the integration component to generate a personalized health assessment for the individual. In one embodiment, the personalized health assessment for the individual comprises health prediction data selected from the group consisting of predicted age of onset for a selected medical condition, predicted health costs for the individual, cost/benefit analysis data of updating phenotypic measurement data associated with the individual, predicted life expectancy of the individual, and combinations thereof. In a preferred embodiment, the received phenotypic measurements are selectively integrated into the at least one age-dependent risk score using the Bayes rule.

In a preferred embodiment, the computing system may comprise a training component operatively connected to the processor and controlled in part by the processor, wherein the training component is configured to generate a plurality of trained genetic risk weights to be used by the genetic risk prediction component in generating the genetic risk scores. In a preferred embodiment, the training component may comprise at least one of (i) a sample training module, (ii) a biological information module, and (iii) a summary module. The training component may be integrated into the genetic risk prediction component or may be a remote component operatively coupled to the genetic risk prediction component,

In a preferred embodiment, the method further comprises receiving, via the input/output device, a plurality of training genetic risk weights associated with a selected medical condition and transmitting the received training genetic risk weights to the training component. In one embodiment, at least one sample parameter for creating a sampling of the received training genetic risk weights is determined by the sample training module. A defined number of the training genetic risk weights to be included in the sampling is selected by the sample training module in accordance with at least one sample parameter. The sampling of training genetic risk weights is then subjected to a resampling process, by the sample training module, to generate trained genetic risk weights. In a preferred embodiment, the sampling of training genetic risk weights is subjected to a penalized regression process to generate the trained genetic risk weights.

In one embodiment, a plurality of biological information associated with the selected medical condition is received by the input/output device and transmitted to the biological information module. In a preferred embodiment, the plurality of received biological information comprises data selected from the group consisting of genic positional annotation data, pleiotropic trait data, gene function data, mutation impact data, predicted functional impact data, genome 3D structure data, and combinations thereof. In one embodiment, at least a portion of the received biological information is selectively incorporated into the trained genetic risk weights by the biological information module to generate enhanced genetic risk weights.

In a preferred embodiment, the method may further comprise receiving a plurality of biological information associated with at least one ancillary medical condition via the input/output device and transmitting the received biological information to the biological information module. At least a portion of the received biological information associated with the at least one ancillary medical condition is selectively incorporated into a least a portion of the plurality of training genetic risk weights by the biological information module to generate enhanced genetic risk weights.

In one embodiment, the enhanced genetic risk weights are then subjected to at least one summary transform function by the summary module to generate a genetic risk score for the individual. In a preferred embodiment, the summary transform function comprises transform functions selected from the group consisting of linear transform functions, exponential transform functions, polynomial transform functions, and combinations thereof.

It is to be understood that the received genetic risk weights may be subjected to one or more of the sample training module, the biological information module, and the summary module, in any combination, to generate a genetic risk score for the individual. For example, in one embodiment, biological information may be directly incorporated into the received genetic risk weights, without first subjecting the received genetic risk weights to a resampling process. In yet another embodiment, the received genetic risk weights may first be trained, and then the trained genetic risk weights are subjected to a summary transform function without incorporating biological information.

In one embodiment of the present disclosure, the method may further comprise receiving, via the input/output device, a plurality of updated phenotypic measurement data associated with the individual and transmitting the updated phenotypic measurement data to the integration component. At least a portion of the update phenotypic measurements is selectively integrated into the at least one age-dependent genetic risk score by the integration component to generate an updated personalized health assessment for the individual.

In a preferred embodiment, the method may also comprise receiving, via the input/output device, a plurality of genetically informed population normative data associated with at least one medical condition and transmitting the received genetically informed population normative data to the integration component. At least a portion of the genetically informed population normative data is selectively integrated into the at least one age-dependent genetic risk score by the integration component to generate an augmented personalized health assessment for the individual.

In accordance with one embodiment of the present disclosure, there is provided a system for deriving a personalized health assessment for an individual by integrating selected genotypic information with phenotypic measurements associated with the individual. The system may comprise a processor operable to control the computing system, and data storage operatively coupled to the processor, wherein data storage is configured to store a plurality of genotypic information, a plurality of phenotypic measurements, and combinations thereof. The system may also comprise an input/output device operatively coupled to the processor, wherein the input/output device is configured to receive a plurality of data for transmission to the processor and to transmit a plurality of data generated by the processor. The input/output device may be further configured to receive a plurality of trained genetic risk weights associated with a selected medical condition, a plurality of germline genetic information associated with the individual, and a plurality of phenotypic measurement data associated with the individual. The computing system may also comprise a genetic risk prediction component operatively connected to the processor and controlled in part by the processor, wherein the genetic risk prediction component is configured to generate a plurality of genetically defined lifetime risks of having a plurality of diseases, and an integration component operatively coupled to the processor and controlled in part by the processor, wherein the integration component is configured to integrate genotypic information with phenotypic measurements.

In one embodiment, the input/output device may be operable to: (i) receive a plurality of trained genetic risk weights associated with at least one selected medical condition and transmit at least a portion of the trained genetic risk weights to the genetic risk prediction component, (ii) receive a plurality of germline genetic information associated with the individual and transmit the received germline genetic information to the genetic risk prediction module, and (iii) receive a plurality of phenotypic measurement data associated with the individual and transmit the received phenotypic measurement data to the integration component.

In an embodiment, the genetic risk prediction component may be operable to: (i) receive at least a portion of the trained genetic risk weights from the input/output device, and (ii)receive at least a portion of the germline genetic information from the input/output device and subject at least a portion of the received germline genetic information to a genetic risk prediction function using at least a portion of the trained genetic risk weights to generate at least one age-dependent genetic risk score for the individual.

In another embodiment, the integration component may be operable to: (i) receive at least a portion of phenotypic measurement data associated with the individual, and (ii) selectively integrate at least a portion of the received phenotypic measurement data into the at least one age-dependent genetic risk score to generate a personalized health assessment for the individual. In a preferred embodiment, the system may further comprise a training component operatively connected to the processor and controlled in part by the processor, wherein the training component is configured to generate a plurality of trained genetic risk weights. In one embodiment, the input/output device is further operable to: (i) receive a plurality of training genetic risk weights associated with the at least one selected medical condition and transmit at least a portion of the plurality of training genetic risk weights to the training component, and (ii) transmit at least a portion of the trained genetic risk weights to the genetic risk prediction component for use in generating the at least one age-dependent genetic risk score. In one embodiment, the training component may be operable to: (i) receive at least portion of the plurality of training genetic risk weights from the input/output device and subject at least a portion of the plurality of training genetic risk weights to at least one training function to generate trained genetic risk weights, and (ii) transmit at least a portion of the trained genetic risk weights to the input/output device.

In accordance with one embodiment of the present disclosure, there is provided a method for deriving a genetic risk score for an individual via a computing system. The computing system may comprise a processor operable to control the computing system, data storage operatively coupled to the processor, wherein data storage is configured to store a plurality of genotypic information, and an input/output device operatively coupled to the processor, wherein the input/output device is configured to receive a plurality of data for transmission to the processor, wherein the input/output device is configured to transmit a plurality of data generated by the processor. The computing system may also comprise a genetic risk prediction component operatively connected to the processor and controlled in part by the processor, wherein the genetic risk prediction component is configured to generate a plurality of genetically defined lifetime risks of having a plurality of diseases.

In one embodiment, the method for deriving a genetic risk score comprises receiving, via the input/output device, a plurality of trained genetic risk weights associated with a selected medical condition and transmitting the received trained genetic risk weights to the genetic risk prediction component. The method may further comprise receiving, via the input/output device, a plurality of germline genetic information associated with the individual and transmitting the received germline genetic information to the genetic risk prediction component. The method may also comprise subjecting, via the genetic risk prediction component, at least a portion of the received germline genetic information to a genetic risk prediction function using at least a portion of the plurality of trained genetic risk weights to generate at least one age-dependent genetic risk score for the individual.

In a preferred embodiment, the computing system may further comprise the method may further comprise an integration component operatively coupled to the processor and controlled in part by the processor, wherein the integration component is configured to integrate genotypic information with phenotypic measurements. The method may also comprise receiving a plurality of phenotypic measurements associated with the individual via the input/output device and transmitting the received phenotypic measurements to the integration component. In one embodiment, at least a portion of the received phenotypic measurements is selectively integrated into the at least one age-dependent genetic risk score by the integration component to generate a personalized health assessment for the individual.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIGS. 1A-C are an overview of exemplary systems and methods for deriving personalized health assessment through integrating genetic information and phenotypic measurements according to the present invention.

FIG. 2 is a block diagram illustrating an example system environment for deriving personalized health assessment through integrating genetic information and phenotypic measurements according to the present disclosure.

FIG. 3 illustrates a simulation based on Alzheimer's disease genetic data using the training and testing processes according to the method of the present disclosure.

FIG. 4 illustrates the quantile-quantile plots of Alzheimer's disease GWAS conditioned on lipid profiling according to the method of the present disclosure.

FIG. 5 illustrates the risk stratification of testing based on polygenic component only according to the method of the present disclosure.

FIG. 6 illustrates a quantile-quantile plot by conditioning on information of genomic regulator machinery according to the method of the present disclosure.

FIG. 7 illustrates a comparison of the performance of each different test for Alzheimer's disease, using PHS as a reference base according to the method of the present disclosure.

FIG. 8 illustrates the benefit of having a genetically adjusted PSA level according to the method of the present disclosure.

FIG. 9 illustrates the benefits to predicting future risks for an individual based on having additional tests given prior available information according to the method of the present disclosure.

FIG. 10 illustrates the results from updating personalized health risk after additional phenotypic measurements

FIG. 11 illustrates the Positive Predictive Value for performing additional tests on an individual according to the method of the present disclosure.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are signify both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer- readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

In various implementations, there may be provided a system and methods for integrating phenotypic measurement data associated with an individual with the individual's germline genetic information. The integration approach may provide short term/long term health prediction, evaluation of specific tests, clinical or medical decision support, and life actuarial calculation.

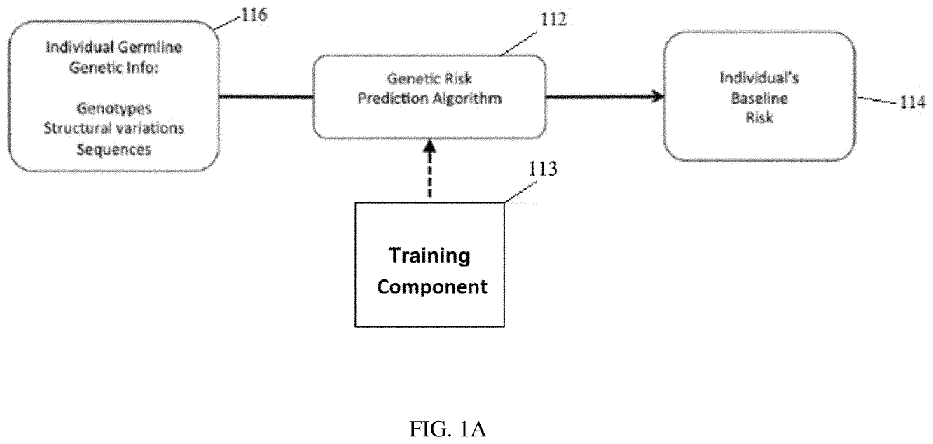

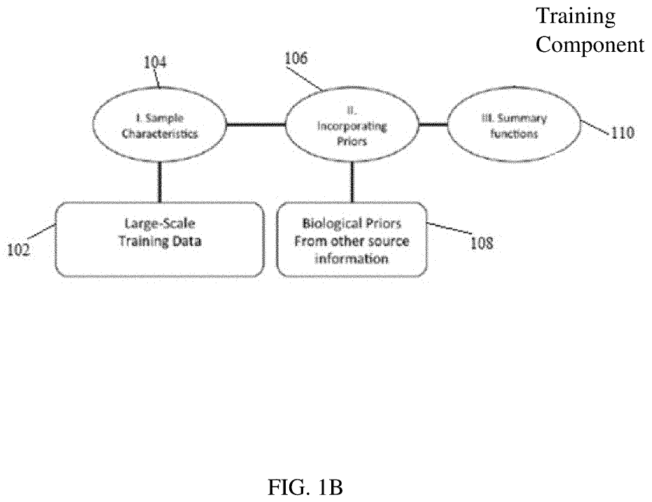

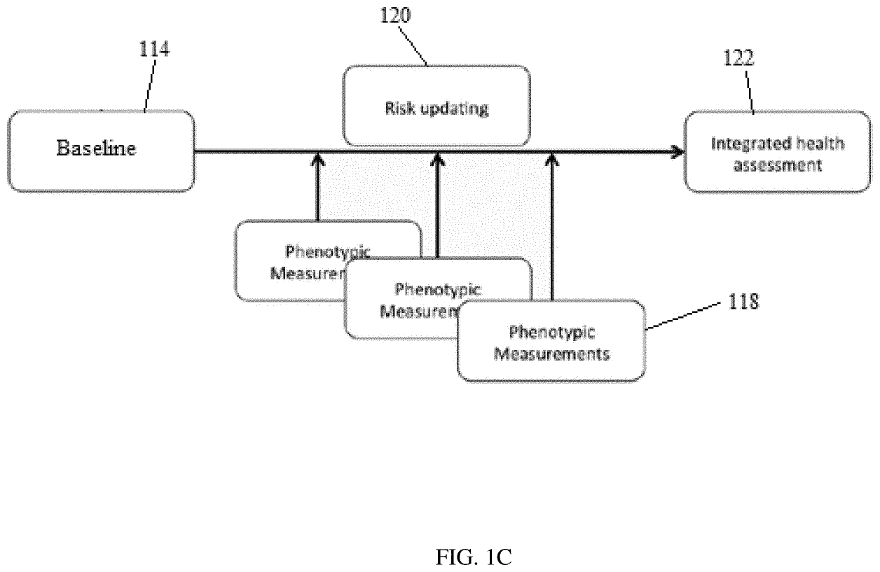

In some embodiments, the present invention provides processes, systems, and methods for providing health assessments through combining genotypic information and phenotypic measurements. FIGS. 1A, 1B, and 1C provide an overview 100 of exemplary systems and methods for deriving a personalized health assessment through integrating genetic information and phenotypic measurements according to the present invention. The process comprises obtaining a plurality of genetic information, wherein the genetic information includes at least one of sequenced genomic data, genotyped calls, imputed genetic data, structural variations, copy number variations, and combinations thereof. In a preferred embodiment, as shown in FIGS. 1A and 1B, the genotypic information may be obtained from large scale genome-wide association studies (GWAS) 102 for the disease and/or condition of interest. In a preferred embodiment, the genotypic information obtained from GWAS comprises a plurality of genetic risk weights that summarize the overall disease risk given a set of genetic variants.

In one embodiment, the genotypic information may comprise a plurality of trained genetic risk weights associated with one or more selected medical conditions and a plurality of germline genetic information associated with the individual and transmitting the received germline genetic information to the genetic risk prediction component. The germline genetic information as shown at 116 includes, but is not limited to, genotypes, structural variations, sequences, and the like. The baseline risk may be updated with phenotypic measurements. At least a portion of the received germline genetic information, is subjected to a genetic risk prediction algorithm or genetic risk prediction component as shown at 112 using at least a portion of the plurality of trained genetic risk weights to generate at least one age-dependent genetic risk score or baseline risk for the individual as shown at 114.

The method further comprises obtaining phenotypic information as shown at 118, wherein the phenotypic information may include, but is not limited to, biomedical or health care records, bioassays, medical imaging data, cognitive performance data and/or neuropsychological test data, behavioral assessments, blood and/or metabolic test data, physiologic data, and the like, and combinations thereof. The phenotypic information is integrated with the at least one age-dependent genetic risk score by an integration component using updating rules as shown at 120 to generate a personalized heath assessment shown at 122. The phenotypic information may be integrated with the predicted genetic risk using the Bayes rule, information theory, joint modeling, and the like. Additional phenotypic information for an individual, such as results from later medical tests, may be incorporated to update the personalized health assessment.

In one embodiment according to the present disclosure, the genotypic information may further comprise a plurality of training genetic risk weights associated with one or more selected medical conditions. At least a portion of the training genetic risk weights are subjected to at least one training process by a training component 113 to generate a plurality of trained genetic risk weights to be used by the genetic risk prediction component in generating the genetic risk scores. In a preferred embodiment, the training component 113 may comprise at least one of a sample training module 104, a biological information module 106, and a summary module 110.

In a preferred embodiment, at least a portion of the received training genetic risk weights are trained by a sample training module to boost the predictive accuracy as shown at 104. The training genetic risk weights are subjected to a resampling process to generate trained genetic risk weights. In a preferred embodiment, the sample case-controls from GWAS for the condition of interest are subjected to a penalized regression to reduce the variation in the sample case-controls and improve the predictive performance.

In one embodiment, a plurality of biological information associated with the selected medical condition is received and transmitted to a biological information module. At least a portion of the received biological information is selectively incorporated into the trained genetic risk weights by the biological information module to generate enhanced genetic risk weights. In a preferred embodiment, the trained genetic risk weights are conditioned using statistical biological information from prior studies to boost the predictive accuracy of the genetic risk weights as shown at 106. The biological prior information shown at 108 may include, but is not limited to, genic annotations about the regulatory machinery of the human genome, biological pathways of gene, structural information about the human genome, algorithm predicted functional impact of genetic mutations, and combinations thereof.

In one embodiment, the enhanced genetic risk weights are then subjected to a summary transform function by the summary module to pool the estimated weights for genetic variants into a single genetic risk score for the individual as shown at 110. In a preferred embodiment, the summaries may be linear, non-linear, data-driven, and the like. In one embodiment, the genetic risk prediction algorithm uses the genetic risk weights obtained from the training component 113 to summarize the germline genetic information from a given individual and generate the at least one age-dependent genetic risk score.

It is to be understood that the received genetic risk weights may be subjected to one or more of the sample training module, the biological information module, and the summary module, in any combination, to generate a genetic risk score for the individual. For example, in one embodiment, biological information may be directly incorporated into the received genetic risk weights, without first subjecting the received genetic risk weights to a resampling process. In yet another embodiment, the received genetic risk weights may first be trained, and then the trained genetic risk weights may be subjected to a summary transform function without incorporating biological information.

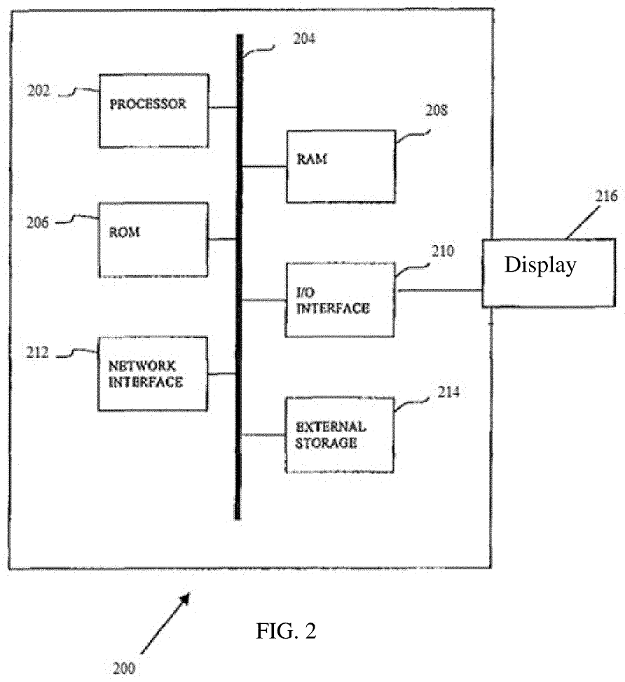

FIG. 2 is a high-level block diagram illustrating an example system environment for deriving personalized health assessment through integrating genetic information and phenotypic measurements according to the present disclosure. The system 200 is shown as a hardware device, but may be implemented in various forms of hardware, software, firmware, special purpose processors, or a combination thereof. Some embodiments are implemented in software as a program tangibly embodied on a program storage device. By implementing with a system or program, semi-automated or automated workflows are provided to assist a user in generating personalized health assessments.

The system 200 is a computer, personal computer, server, PACs workstation, mobile computing device, imaging system, medical system, network processor, network, or other now know or later developed processing system. The system 200 includes at least one processor 202 operatively coupled to other components via a system bus 204. The processor 202 may be, or may comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU). In one embodiment, the processor 202 may be implemented on a computer platform, wherein the computer platform includes an operating system and microinstruction code. The various processes, methods, acts, and functions described herein may be either part of the microinstruction code or part of a program (or combination thereof) which is executed via the operating system as discussed below.

The other components include memories (ROM 206 and/or RAM 208), a network access device 212, an external storage 214, an input/output device 210, and a display 216. Furthermore, the system 200 may include different or additional entities.

The input/output device 210, network access device 212, or external storage 214 may operate as an input operable to receive at least a portion of at least one of the genotypic information and the phenotypic measurements. Input may be received from a user or another device and/or output may be provided to a user or another device via the input/output device 210. The input/output device 210 may comprise any combinations of input and/or output devices such as buttons, knobs, keyboards, touchscreens, displays, light-emitting elements, a speaker, and/or the like. In an embodiment, the input/output device 210 may comprise an interface port (not shown) such as a wired interface, for example a serial port, a Universal Serial Bus (USB) port, an Ethernet port, or other suitable wired connection. The input/output device 210 may comprise a wireless interface (not shown), for example a transceiver using any suitable wireless protocol, for example Wi-Fi (IEEE 802.11), Bluetooth®, infrared, or other wireless standard. In an embodiment, the input/output device 210 may comprise a user interface. The user interface may comprise at least one of lighted signal lights, gauges, boxes, forms, check marks, avatars, visual images, graphic designs, lists, active calibrations or calculations, 2D interactive fractal designs, 3D fractal designs, 2D and/or 3D representations, and other interface system functions.

The network access device 212 allows the computing system 200 to be coupled to one or more remote devices (not shown) such as via an access point (not shown) of a wireless network, local area network, or other coupling to a wide area network, such as the Internet. In that regard, the processor 202 may be configured to share data with the one or remote devices via the network access device 212. The shared data may comprise, for example, genetic information, phenotypic information, genetic risk prediction data, and the like. In various exemplary embodiments, the network access device 212 may include any device suitable to transmit information to and from another device, such as a universal asynchronous receiver/transmitter (UART), a parallel digital interface, a software interface or any combination of known or later developed software and hardware. The network access device 212 provides a data interface operable to receive at least a portion of at least one of the genotypic information and the phenotypic measurements.

The processor 202 has any suitable architecture, such as a general processor, central processing unit, digital signal processor, application specific integrated circuit, field programmable gate array, digital circuit, analog circuit, combinations thereof, or any other now known or later developed device for processing data. The processor 202 may be a single device or include multiple devices in a distributed arrangement for parallel and/or serial processing. Likewise, processing strategies may include multiprocessing, multitasking, parallel processing, and the like. A program may be uploaded to, and executed by, the processor 202.

The processor 202 performs the workflows, data manipulation of the genetic information, integration of phenotypic measurements with the genotypic information and/or other processes described herein. The processor 202 operates pursuant to instructions. The genotypic information and the phenotypic measurements may be stored in a computer readable memory, such as the external storage 214, ROM 206, and/or RAM 208. The instructions for implementing the processes, methods and/or techniques discussed herein are provided on computer-readable storage media or memories, such as a cache, buffer, RAM, removable media, hard drive or other suitable data storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system. Because some of the constituent system components and method acts depicted in the accompanying figures may be implemented in software, the actual connections between the system components (or the process steps) may differ depending upon the manner of programming.

The external storage 214 may be implemented using a database management system (DBMS) managed by the processor 202 and residing on a memory, such as a hard disk, RAM, or removable media. Alternatively, the storage 214 is internal to the processor 202 (e.g. cache). The external storage 214 may be implemented on one or more additional computer systems. For example, the external storage 214 may include a data warehouse system residing on a separate computer system, a PACS system, or any other now known or later developed storage system.

### A. Augmenting the Performance of Genetic Risk Prediction

The system and method of the present disclosure use three different modules to improve the performance of age-dependent risk prediction based on genetic information. One module exploits the characteristics of the training sample, boosting predictive accuracy through efficiently using time-dependent information. The second module incorporates the biological priors into the prediction model, borrowing statistical strength from other large-scale genetic studies. The third module tackles the need for summary function that effectively pooling the estimated weights for genetic variants into one single risk score. Each of the modules may be used independently as each module has the functionality to boost the predictive performance of the genetic risk scores. The system and method are not just based on the genetic information from case-controls, but integrate available relevant information to boost the predictive power based on genetics.

### 1. Cohort Characteristic Sensitive Training

Many diseases and human traits have a strong time component. For instance, people inherited with APOE ε4 risk allele would tend to have an earlier age-at-onset for Alzheimer's disease. Incorporating this time-dependent feature into the model has shown to improve the risk prediction. However, most of large-scale GWAS are based on the case-control design with convenient sampling. Therefore, the conventional genetic risk prediction based on logistic regression or probit regression become agnostic to the age-dependent process, and fail to approximate the incidence rate due to the loss of density sampling scheme. Although recent studies have demonstrated the benefit of analyzing genetic risk in the context of survival analysis, it is unclear how the marginally sampled case-control can be helpful for training a well-generalized genetic risk predictor. In particular, the unknown sampling probability among controls disrupts the presumed characteristics of the risk set, which are those who potentially can be inflicted by the disease but not yet, which is the fundamental building block of survival analysis.

To illustrate this more concretely, the genetic risk prediction with a time-dependent feature for individual j (. j=1, 2, . . . , n) and m genetic factor can be formulated as:

Φ(Tⱼ, Dⱼ)⁻¹=α+βₘKₘ(Gⱼ) (1)

In the above equation, D is the binary outcome, T is when D happens, Φ(.)⁻¹ maps the linear sum to appropriate non-linear function (e.g., Weibuil or exponential function), and G is an individual's genotypes. The weights needed to be estimated are βₘ. K is a kernel function to sum over the input G. Conventionally, the function K is a linear function, hence making the right-hand side of the formula a simple linear sum of all weighted genetic effects.

To identify the weight for a given genetic factor, m, the convention of Cox proportional hazard model seeks to maximize the differences between the risk of those who happen to have the disease at given time T and average risk of the risk set:

$${\hat{β}}_{m} = {arg max}_{{β}_{m}} {\sum }_{j ∈ D} {β}_{m} K ( {G}_{j} ) - log ( {\sum }_{i ∈ R ( {T}_{j} )} exp ( {β}_{m} K ( {G}_{i} ) ) ) \qquad ( 2 )$$

The risk set, R(Tⱼ), represents those individuals who still have probability to get the outcome in the cohort, before they dropped out cohort or have the eventual outcome, D, happen. From equation (2), it should be noted that the estimation of βₘ is dependent on how the risk set is constituted. If the risk sets have more high-risk individuals, the estimation would tend to reduce the estimated value of β. Typically GWAS, wherein large sample sizes are mandated for any polygenic model, oversamples with high-risk individuals without properly matching the sampling among controls. Therefore, the utility of a training survival model with GWAS data was unclear despite the empirical utility it has demonstrated.

The method of the present disclosure exploits the concept of risk set to tune the training efficiency for predictive performance. Our method does not treat the sampling proportion of case-controls from GWAS as it is. Instead, by tuning the estimation through resampling the proportion of case-controls in the risk set, the generalizability of the predictive model may be boosted. In this context, the training scheme is reformulated with marginally sampled case-control GWAS as penalized regression. With some linear algebra, the optimization equation (Error! Reference source not found.) may be rearranged into:

$$\begin{matrix}{\overline{β}}_{m} = {arg max}_{β} {\sum }_{j ∈ D} {β}_{m} K ( {G}_{j} ) - log ( {\sum }_{i ∈ {R}_{controls} ( {T}_{j} )} w exp ( {β}_{m} K ( {G}_{i} ) ) + \\ {\sum }_{i ∈ {R}_{cases} ( {T}_{j} )} exp ( {β}_{m} K ( {G}_{i} ) ) ) \\ = {arg max}_{{β}_{m}} log l ( {β}_{m} ) - P ( {β}_{m} , w )\end{matrix}$$

The penalty function, P, is a function of β and the sampling weights, w, which is partially known from the mixing proportion of cases and controls in the data. The estimated weight, βₘ, is a biased estimate regarding to the true β because of the unknown sampling probability in the risk set. However, the proportion of cases and controls in the training risk set may be tuned to change the amount of penalty. Therefore, by manipulating the proportion of cases and controls in the training data, we may trade bias with model variance, improving the prediction performance accordingly.

To demonstrate the validity of our formulation, we simulated the training and testing processes using a realistic large-scale dataset of Alzheimer's genetic cohort. In each simulation, we randomly sampled 10,000 individuals from the cohort, while varying proportions of cases and controls in the training, and then tested the model performance in the independent dataset (n=16000).

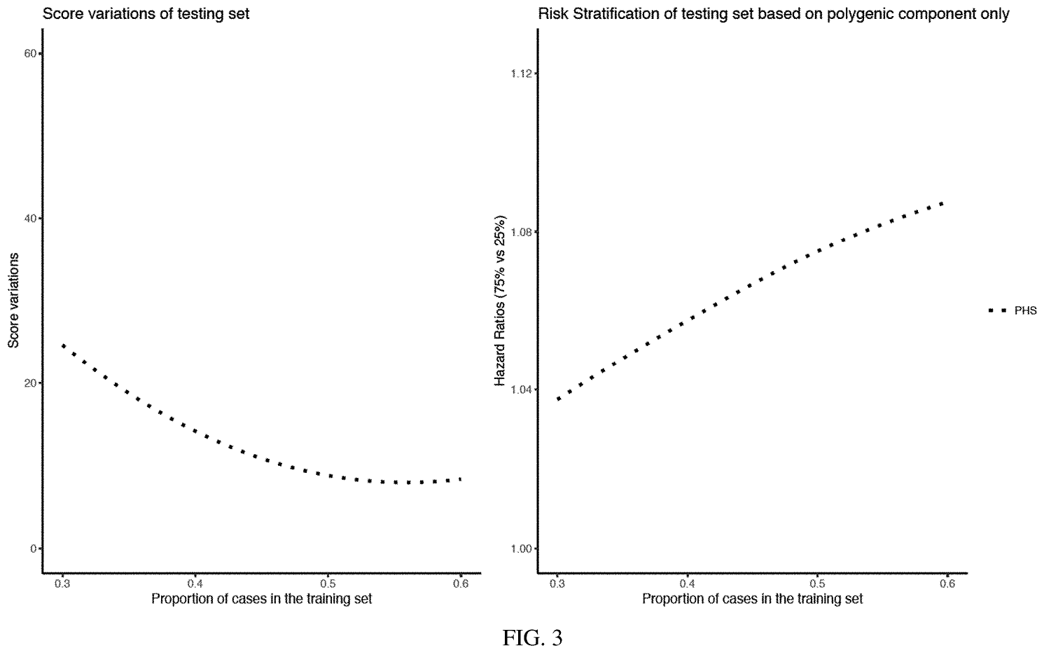

FIG. 3 illustrates the simulation 300 based on Alzheimer' s disease genetic data. The left panel 302 is the effect of shrinkage on the magnitude of the score. The right panel 304 is the prediction performance in the independent dataset.

As FIG. 3 demonstrates, the variations of β decreased and the performance increased for the testing set despite the number of training samples being fixed and only varying the proportion of cases in the training samples. Tuning the proportion of cases and controls in the training set imposes an implicit penalty function, trading some bias while evidently reducing the model variation. This shows our approach matches with the conceptualization of penalized regression, reducing the variation of the model (reduced overall magnitude of the score), while improving the generalization (predictive accuracy).

Our approach provides a promising improvement over the conventional approach on training genetic risk models based on GWAS. The penalized regression based on resampling proportion is just one way to exploit the risk set. For example, the risk set may be pre-determined through empirical Bayes estimation or plugging in the results from a previous epidemiological survey. The estimation may use more than one sampling process, such as a jackknife estimator that averages multiple instantiations.

### 2. Improving Estimation Based on Prior Information

For human complex traits with multiple genes involved, the per genetic variant effect is hard to detect due to limited statistical power. This impacts the accuracy of genetic risk prediction, because the performance of the model is dependent upon the reliability of the estimation on per variant effect. One way to boost reliability is to borrow the statistical strength from other genetic studies. For instance, it is known that the effect sizes of genetic variant are correlated among causally related traits. Thus, we gain additional information about a given genetic variant if we conditioned based on results from other studies.

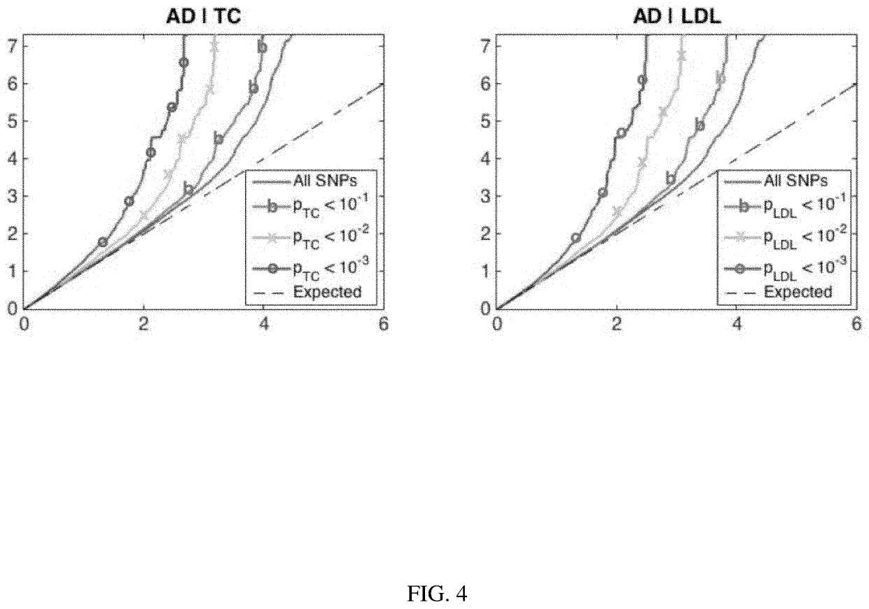

FIGS. 4A-B illustrate this conditional phenomenon. FIGS. 4A-B illustrate the quantile-quantile plots of Alzheimer's disease GWAS conditioned on lipid profiling GWAS. FIG. 4A illustrates Alzheimer's disease GWAS condition on total cholesterol. FIG. 4B illustrates Alzheimer's disease GWAS condition on low density lipoprotein. The quantile-quantile plots characterize the effect size distribution per genetic variant effect. The dashed lines are the expected null distribution, meaning the p-values of a given GWAS distributed are as random as uniform distribution. When conditioned on the GWAS of lipid profiles, which is associated with the etiology of Alzheimer's disease, the signals of GWAS on Alzheimer's disease are enriched, as the flex upward of the quantile-quantile plot shown.

Previous studies have demonstrated that this phenomenon is relatively ubiquitous among human complex traits and may be exploited to boost the statistical power for estimating the per genetic variant effect. Nevertheless, it was unclear if incorporating this conditional information into the genetic risk prediction would improve the model performance.

The present disclosure provides a method to incorporate conditional information into our genetic risk prediction. In the context of age-dependent process, we are aiming to obtain the estimation on genetic effects through a linearly transformed model, as equation (2). Assuming we obtain the linearly transformed liability for each individual j as ηⱼ, the least squared solution may be expressed as

\hat{β}ₘ=(K(G)K(G)′)⁻¹K(G)\vec{η} (3)

For the survival analysis, we can approximate individual's η using Martindale residuals after regressing out the nuisance factors, such as gender, study sites, and genetic ancestries as shown in equation (4). In equation (4) Xⱼ is the covariates and γ is the corresponding effects.

\hat{η}ⱼ=Dⱼ−\hat{Φ}( [symbol] ) (4)

Now we further assume the kernel summary of the genetic function is distributed as N(0,1) and the corresponding effect size β is distributed as N(0,σ²ₘ). In this formulation, if we know the σ²ₘ beforehand, then we can approximate the maximum a posteriori solution for β as shrinkage estimates

$$\begin{matrix}{\overline{β}}_{m} \approx ( 1 - {κ}_{m} ) {\hat{β}}_{m} & ( 5 ) \\ where & \\ {κ}_{m} = \frac{1}{1 + N {δ}^{- 2} {σ}_{m}^{2}} & ( 6 )\end{matrix}$$

There are many ways to obtain the σ²ₘ as the prior information. Different methods have been described elsewhere, such as PCT Patent Application No. PCT/US2014/011014, incorporated herein by reference. For example, we can obtain the expected values of σ²ₘ using linkage disequilibrium (ld) score regression, conditioning on results from other genetic studies. For summary statistics with respect trait K, there exists a linear relationship between the observed effect size in a current trait and the ld weighted effect sizes from trait K.

$$E [ {σ}^{2} \vert {χ}_{k}^{2} ] = {γ}_{0} + {γ}_{k} {\sum }_{l} l {d}_{l} {χ}_{k}^{2} \qquad ( 7 )$$

By plugging in the expected σ²ₘ to the equation (6) as an empirical Bayes estimate, the shrinkage estimates may be obtained accordingly. With this, obtaining the prior informed estimation of β becomes very fast, making the whole genome scan in large-scale GWAS feasible. To demonstrate the validity of this approach, the same simulation procedures discussed above with respect to Cohort Character Sensitive Training were used. For comparison purposes, the polygenic hazard score (PHS), our polygenic model without priors, and the polygenic risk score (PRS), the traditional polygenic risk model, were included. The prior informed estimation is used in the enriched PHS.

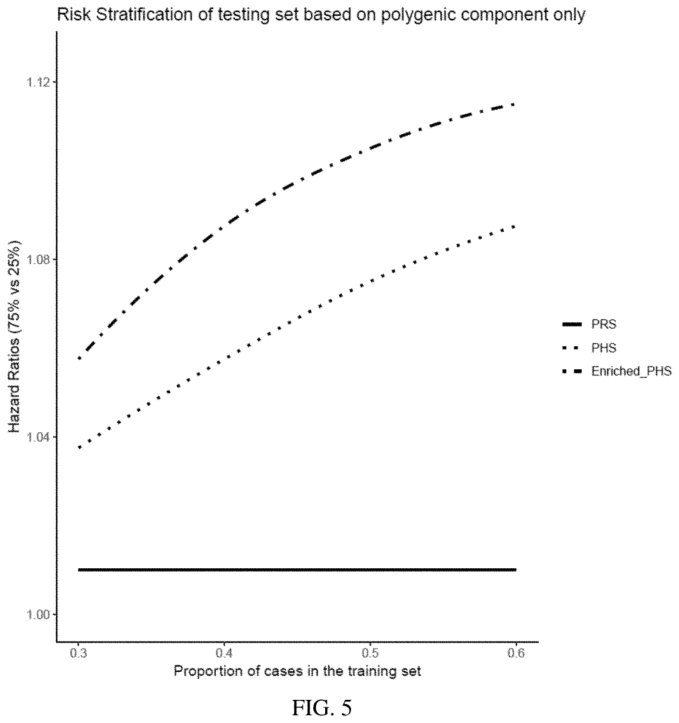

FIG. 5 illustrates the risk stratification of testing based on polygenic component only. As shown in FIG. 5, the enriched PHS maintained the benefit of varying the proportion of cases involved in the training, as did the PHS obtained with respect to Cohort Character Sensitive Training. Moreover, additional shrinkage from priors provide a further performance books. On the other hand, the traditional PRS had very limited performance in this instance.

It is to be understood that the process of incorporating prior information is not limited to the shrinkage estimates demonstrated. It may be achieved through either full Bayesian approach, such as MCMC, or weighted regression through penalized weightings.

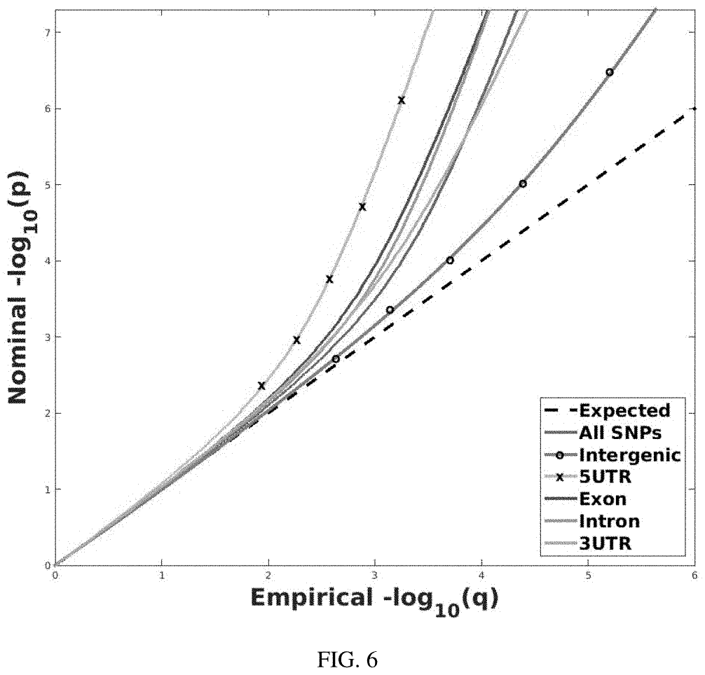

One process to obtain the prior information is to use the methods set forth in PCT Patent Application No. PCT/US2014/011014. In short, the methods model the distribution of effect sizes of a given GWAS based on observed patterns from other genetic studies. FIG. 6 illustrates a quantile-quantile plot by conditioning on information of genomic regulator machinery. The observed patterns may be gained from studies on genomic regulatory machinery, such as positional annotations about promoters, enhancers, and distance to gene bodies as illustrated in FIG. 6. It may also be gained from pleiotropic traits, meaning traits that share common genetic factors, as demonstrated in FIG. 4. In PCT Patent Application No. PCT/US2014/011014, the main source of prior information is suitably gained from 1) genic annotations and 2) pleiotropic effects from other traits.

Any genomic features having impact on gene expression may be found to have traceable influence on complex traits. Hence, the prior information for effect estimation may also include, but is not limited to:
1. Effect sizes from the GWAS results of pleiotropic traits;
2. Functional annotations of given variants;
3. Gene functions in the biological pathways;
4. Mutation impact on the molecular structure;
5. Model predicted functional impact;
6. Higher order mutual relationships across geneses, such as biological networks;
7. Genome 3D structures.

### 3. Functions for Deriving Risk Scores

The transform function Φ(.) and kernel function K(.) provides the flexibility for our algorithm to maintain the computational efficiency of a linear model, while capturing all potential non-linear relationships between genes and traits. As discussed above, a transform function Φ(.), such as e Weibull or the exponential function may be used for survival analysis.

For continuous outcomes, such as measurements from memory tests, the model is extended as linear mixed effects model.

Φ(Δ_{t,t+1})⁻¹=α+βₘKₘ(Gⱼ)+ϵⱼ (8)

where Δ is the differences of continuous outcome between time t and t+1. [symbol] represents the random errors. Meanwhile, the kernel function may be specified as a basis function to summarize the non-linearity of a given genetic variant and its correlations with neighboring variants. Given the basis function as a matrix W, genetic effect may be expressed as

\tilde{β}K(G)=\vec{η}XWW′X′ (9)

where η is the Φ(.) transformed continuous liability value of n individuals as an nx1 vector. X is a nxm matrix that contains m genetic variants, usually genetic variants within the 150 Kb to 1 Mb regions. The basis function transforms m genotype dosages into kernels, which may be linear, polynomial, or another basis. If we use the linear kernel, the result is identical to the univariate βₘ mentioned earlier, and we may incorporate the priors σ²ₘ as the nominator in the kernel function. All the results obtained as set forth above are based on the linear kernel function, with and/or without priors.

The flexibility of our formulation also enables further extensions on the training algorithm. As discussed above, the theoretically derived transform function was used. Nevertheless, the transform function may also be generated via a data-driven approach. This includes, but is not limited to, machine learning methods, such as deep learning, kernel machines, support vector machines, random forest, and other related data-driven estimating functions.

### B. Integrating Genotype and Phenotype Information for Risk Prediction

The genetics alone cannot fully reflect an individual's current condition. There is a substantial amount of variation that genetic information cannot characterize. Integrating the genetic information with phenotypic measurements may potentially improve the risk prediction. However, because phenotype measurements, such as magnetic resonance imaging, may be very expensive, it is rare to have a population study that encompass myriad of clinically relevant phenotypic measures. Many tests were examined within a finite sampled cohort, wherein the study population might be very different than the general population seeking medical treatment. Because the effect measures for different tests are based on the contrast within the cohort, each test may have different reference points. Such difference in reference points becomes problematic when combining different tests to infer a personalized health status.

In this context, even though the genetic risk prediction cannot fully characterize individual's risks, it may serve as a reference a with biological anchor. Because germline genetic variants are invariant across lifespan and have consistent effect in the population level, genetic information can provide a personalized reference point. As such, individual test results may be compared with those who inherited with similar genetic profiles. The genetic prediction may serve as a biological anchor, homogenizing the comparisons across diverse studies, making the combination of different tests possible.

To demonstrate this principle, the Alzheimer' s Dementia Neuroimaging Initiative (ADNI) data was analyzed to see how much risk prediction may benefit from integrating genotype and phenotypes. The test involved determining cerebrospinal fluid β-amyloid (CSF-Abeta), hippocampus occupancy volumes (HOC) from magnetic resonance imaging), and optimal Alzheimer's Dementia (AD) measurements from magnetic resonance imaging (optimal MRI). The CSF-Abeta test results have the fewest subjects (n<200) due to the invasiveness of this test.

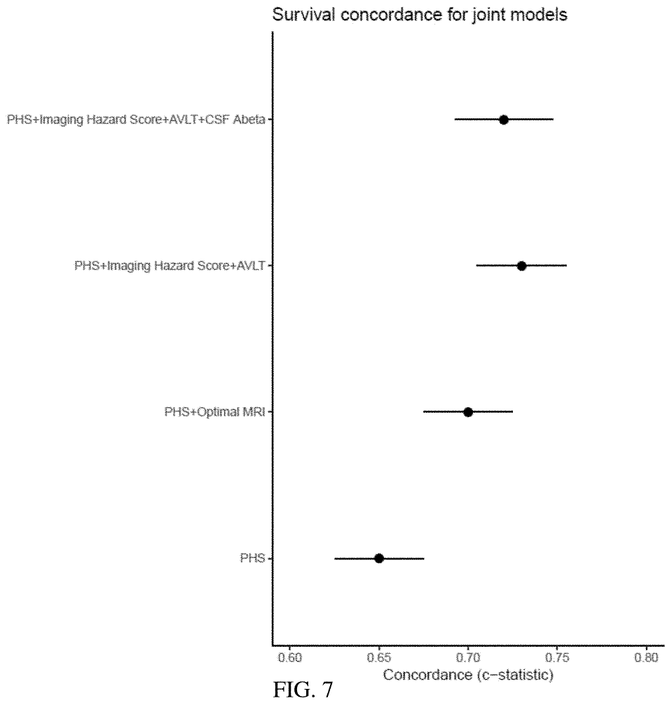

FIG. 7 illustrates a comparison of the performance of each different test for Alzheimer's disease, using PHS as a reference base. As shown in FIG. 7, f only the model performance was examined for each test separately, it seems that CSF-Abeta had the strongest signals for determining the case status. However, when each model was compared with the PHS as reference, the MRI provides better predictive power than CSF-Abeta. This suggests that CSF-Abeta is exaggerated due to biased sample selection, and makes sense as ADNI is not a randomized controlled trial for CSF-Abeta, but a clinician referred sample. Due to the invasiveness of CSF-Abeta, it is often a last resort for clinician to refer patients for diagnostic purpose.

This provides a framework for integrating genotypes and diverse phenotype measurements. The flexibility of this approach enables the integration of many medically relevant measurements, such as, but not limited to:
1. Quantitative measures from magnetic resonance imaging;
2. Neuropsychological tests, such as memory tests;
3. Levels of Prostate Specific Antigen (PSA);
4. Levels of CSF tau and Abeta;
5. Measurements from biochemical assays;
6. Measurements from medical devices, such as optic retinal scan, or DXA (Define?);
7. Gene expression profiles from a biological specimen obtained from an individual under assessment, such as tumor biopsy.

### 1. Consistent Risk Measures

The risk prediction in this model refers to age-dependent disease risks. This may be a survival model for binary disease state or a mixed effects model for continuous measures. As such, available tests are not just used for diagnostic purpose at a current time-point, but may also provide information about potential risk in the near future. Furthermore, it provides to adjustments to the baseline prior probability based on when the tests were done. Then, the health risk may be dynamically updated accordingly in the future when new tests are available.

### 2. Methods for Updating the Risks Given Genotype and Phenotype Data

With respect to risk updating, the following Bayes rule is used to derive the combined report:

$$P ( D = 1 \vert X ) = \frac{P ( X \vert D = 1 ) P ( D = 1 )}{P ( X \vert D = 1 ) P ( D = 1 ) + P ( X \vert D = 0 ) P ( D = 0 )} \qquad ( 10 )$$

It means that the posterior probability to have the disease may be partitioned into the prior probability of having the disease and how likely a person with/without disease would have the same testing values. The process may also be changed to provide a posterior inference. For example, if we have PHS and population disease baseline in a given age, the posterior risk may be updated when an individual receives MRI scans through a series of Bayes calculation

$$P ( D = 1 \vert PHS , Age ) = \frac{P ( P H S \vert D = 1 ) P ( D = 1 \vert Age )}{P ( P H S \vert D = 1 ) P ( D = 1 \vert Age ) + P ( P H S \vert D = 0 ) P ( D = 0 \vert Age )}$$

$$P ( D = 1 \vert MRI , PHS , Age ) = \frac{P ( MRI \vert D = 1 ) P ( D = 1 \vert PHS , Age )}{\begin{matrix}P ( MRI \vert D = 1 ) P ( D = 1 \vert PHS , Age ) + \\ P ( MRI \vert D = 0 ) P ( D = 0 \vert PHS , Age )\end{matrix}}$$

$$P ( D = 1 \vert PHS , Age ) = \frac{P ( P H S \vert D = 1 ) P ( D = 1 \vert Age )}{P ( P H S \vert D = 1 ) P ( D = 1 \vert Age ) + P ( P H S \vert D = 0 ) P ( D = 0 \vert Age )}$$

$$P ( D = 1 \vert MRI , PHS , Age ) = \frac{P ( MRI \vert D = 1 ) P ( D = 1 \vert PHS , Age )}{\begin{matrix}P ( MRI \vert D = 1 ) P ( D = 1 \vert PHS , Age ) + \\ P ( MRI \vert D = 0 ) P ( D = 0 \vert PHS , Age )\end{matrix}}$$

The flexibility of the combination based on Bayes rule enables different strategies to incorporate diverse types of training data. For instance, distribution of PHS was derived from large-scale GWAS and then the baseline risk per genetic risk strata was estimated in the context of survival model. The probability of having the disease in a given time is function of the product of PHS and the population incidence derived from epidemiological survey, as follows:

$$P ( D == 1 \vert PHS , Age ) = \frac{Incidence ( Age ) exp ( PHS )}{C} \qquad ( 11 )$$

where C is the normalizing constant.

In one embodiment, the conditional likelihood may come from different studies. If one study has several relevant medical measures, then the likelihood may be characterized by joint modeling of all variables, ensuring there is no overlapping effect to exaggerate combining all available information at once. If relevant medical measures are only available for a small group individuals or study cohorts, the likelihood function may then be defined separately. As the Bayes rule is used to perform the and genetics already provides a constant biological anchor, the impact of overlapping information is minimized for the combined risk reports.

The flexibility of combining risk allows the use of all available test information for a given individual, whether those tests are performed at once or in the future. For previous test results, the combined risk assessment may be done at once with properly specified joint likelihood. As new tests are performed, previously calculated posteriors serve as priors, and plugged into the equation to determine how much the risk of having disease has been updated changed by the new tests.

### 3. Genetically Informed Population Norms

Even under normal circumstances without any pathological meaning, phenotypic measures may still have substantial variations across individuals. If genetics is utilized to characterize the normal variations across individuals, the diagnostic value of phenotypic measures may be greatly improved. For example, levels of prostate specific antigens (PSA) have substantial heritability such that 30 percent of the variations may be explained by common genetic factors. A polygenic model was trained to predict an individual's PSA level given the individual's genotypes, using publicly available GWAS of PSA level (n=20K) and then calibrated it using healthy subjects from a smaller cohort (n=4K). The predicted PSA level served as a reference point to adjust the observed PSA level. The results were analyzed to determine whether the adjusted PSA level helps to differentiate between high grade tumors and low-grade tumors among patients with prostate cancer (n=30K).

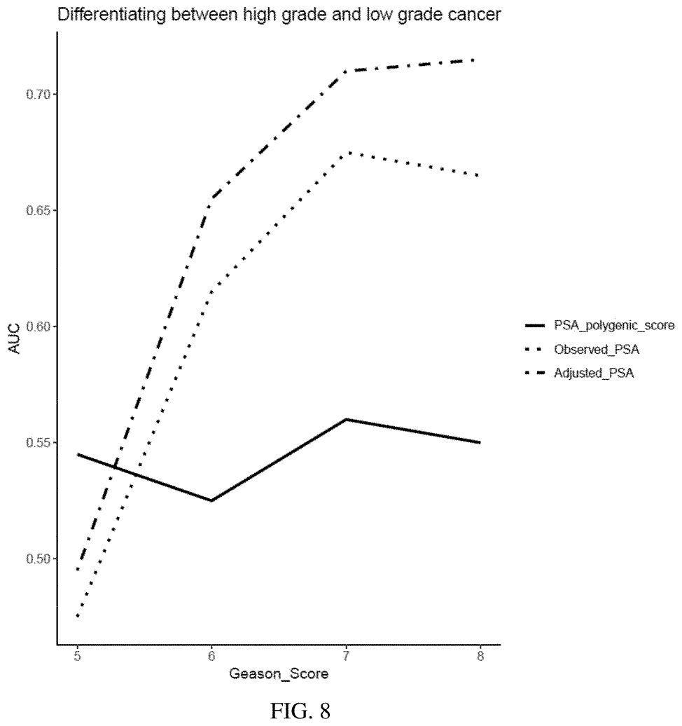

FIG. 8 illustrates the benefit of having a genetically adjusted PSA level. The area under the curve on the y-axis was determined by differentiating high grade versus low-grade tumors based on different thresholds of Gleason scores. The x-axis represents the threshold variance in the Gleason scores to define high grade versus low-grade tumors, providing a systematic evaluation of the model performance. The PSA polygenic score is the genetically predicted PSA level. In our analysis, due to limited availability of summary statistics of PSA GWAS, the genetically predicted PSA level only explained 3 percent of the variance in our normal cohort. Nevertheless, when the PSA level was adjusted by this population norm of PSA, the performance was boosted to such that the AUC value surpassed 70 percent. This demonstrates the utility of having genetically informed population norms in the method of the present disclosure.

Any functions and approaches used in genetic risk prediction may suitably be used in constructing the genetically informed population norms. However, as the goal is to capture the normal variations in the general population, deviations away from the norms used as the primary source for predictive power. For PSA levels, it is the differences between observed and genetically predicted levels that provide the boost in classifying between high grade versus low grade.

Protein level measurements, such as PSA or CSF-Abeta, may be obtained from targeted bio-assays, and as such, the variation of each may be represented by single value. A model for such levels may be built according to the process set forth above, to generate a genetically informed population norm. As the genetically informed norms are used in the context of combined report, it would be preferable to have information that is orthogonal to the genetic risk prediction. With respect to the PSA level illustrated in FIG. 8, the adjusted PSA is indeed orthogonal to prostate PHS. The adjusted PSA has no impact in the prediction of prostate cancer using PHS in an independent large-scale prostate cancer GWAS (n=40K). Nevertheless, it is not necessary to ensure strict independency between genetically informed norms and genetic risk score, as any additional information may improve the prediction.

Measurement from neuroimaging or gene expression from tumors are inherently high dimension, and therefore, the covariance of measures from each modality is important. Therefore, the genetically informed norms need to take such covariance into consideration. This may be achieved either through explicitly modeling the covariance structure, or generating a dynamic atlas to determine the normal template for the genetic information.

### 4. Benefits Gained from Combined Risk Assessment

### a. Predicting Short Term and Long-Term Disease Risks and Prognosis

As the method of the present disclosure positions the risk prediction within the time domain, the assessment does not just provide risk prediction with respect to the current conditions, but also provides future risk predictions for an individual. The probability of having a specific disease or its related prognosis is a function of age and genetics, and be updated according available phenotypic measures. Depending on the property of phenotypic measures and the available training data, phenotypic measures may assist with either short term or long-term risk prediction.

### b. Assessment/Qualification of Value for a Specific Test

The functionality to update risk prediction using the Bayes rule provides the ability to critically examine the value of a specific test. For example, a test to be used as part of a mass public screening must have a good positive predictive value (PPV) to avoid potential over diagnosis. Certain screening tests, such as imaging or biomarker levels, typically have fixed sensitivity (1-false negatives) and specificity (1-false positives). As such the PPV may be dominated by prior prevalence, which may be characterized by either genetic risk prediction or combined risks.

$$PPV = \frac{Sensitivity \cdot P ( D = 1 \vert PHS , M = 1 )}{\begin{matrix}Sensitivity \cdot P ( D = 1 \vert PHS , M = 1 ) + \\ ( 1 - Specificity ) \cdot P ( D = 0 \vert PHS , M = 1 )\end{matrix}} \qquad ( 12 )$$

where M represents the results of the phenotypic measures, in which sensitivity and specificity were defined. The method of the present disclosure also provides the ability to identify subgroups of individuals that may benefit of having a specific test or screening as well as at what age the screening should begin.

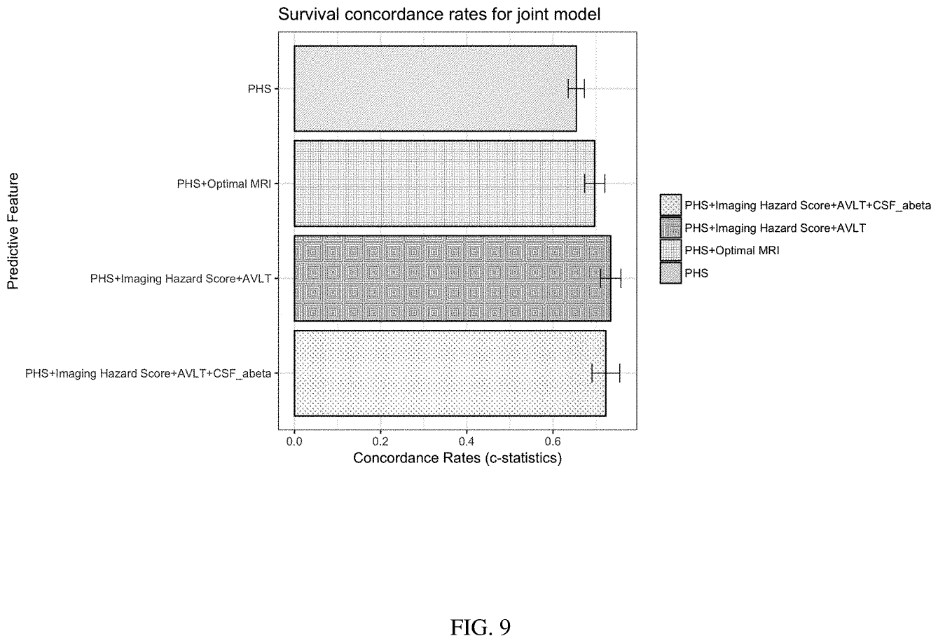

In addition to public screening scenarios, the method of the present disclosure may assist with care pathways in the clinical setting. The benefit of a given test may be evaluated based a personalized risk assessment, genetic scores, and prior tests. As such, clinicians and patients are able to determine whether to proceed with additional tests based on the determined benefit. FIG. 9 illustrates the benefits to predicting future risks for an individual based on having additional tests given prior available information. Using the same ADNI data, the benefit of having CSF-Abeta tested was eliminated if the individual already had genetic risk prediction and MRI scans. Therefore, a patient may avoid additional cost if the patient has low genetic risk, robust brain measures, and good cognitive performance.

### c. Supporting Complex Health Decisions

As the method of the present disclosure unifies all available information into a probabilistic framework, expected values may be assigned accordingly. This enables wider application of medical informatics, such as cost-benefit analysis, life actuarial calculations, and clinical trial estimations. For example, the cost and benefit may be weighed by assigning monetary values for the potential cost of successful intervention and probable complications, as

E[Cost]=E[Cost|S=1]P(S=1|MRI, PHS)+E[Cost|S=0]P(S=0|MRI, PHS)

E[Cost|S=1]=∫ Cost(x)P(S=1|x)dx

E[Cost|S=0]=∫ Cost(x)P(S=0|x)dx

where S is the indicator whether the intervention is successful or results in complications.

The expected values of each different scenario are derived through integrating relevant cost for a given intervention x. This may be further expanded to calculate the potential medical expenses given all possible outcomes in a given age. Further, the integrated genetic risk predictions allow for efficient selection in participants in a clinical trial, either for purposes of reducing cost, increasing statistical power, controlling confounding factors, or identifying outliers.

In addition, the same principle may be applied to domains other than clinical settings, such as life actuarial calculations. The risk probability is defined with age-dependent component. Therefore, the expected age for potential outcome may be calculated. Other costs, such as expected health costs across a disease domain, lost productivity, and the like, may also be calculated.

### EXAMPLE 1

### Updating Personalized Health Risk after Additional Phenotypic Measurements

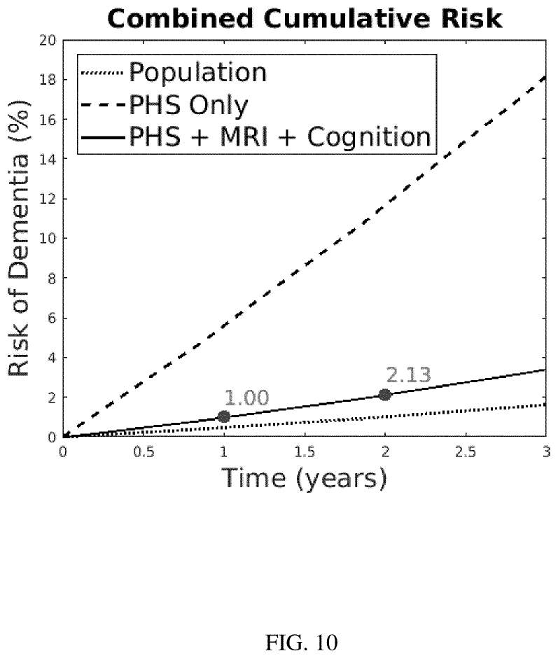

To demonstrate the utility of personalized health assessment using both genotype and phenotypic measurements, we examined the expected risk of having Alzheimer' s disease in the longitudinal follow-up data from Alzheimer's Dementia Neuro Imaging (ADNI) cohort. One subject from the ADNI was predicted as high-risk given the individual's PHS. FIG. 10 illustrates the results of such analysis. As shown in FIG. 10, the individual has relatively robust results from cognitive tests and the scans from magnetic resonance imaging shows intact hippocampus volume, therefore, the resulting risk was much lower than the prediction based on germline genetics.

### EXAMPLE 2

### Evaluating the Benefit of Novel Tests

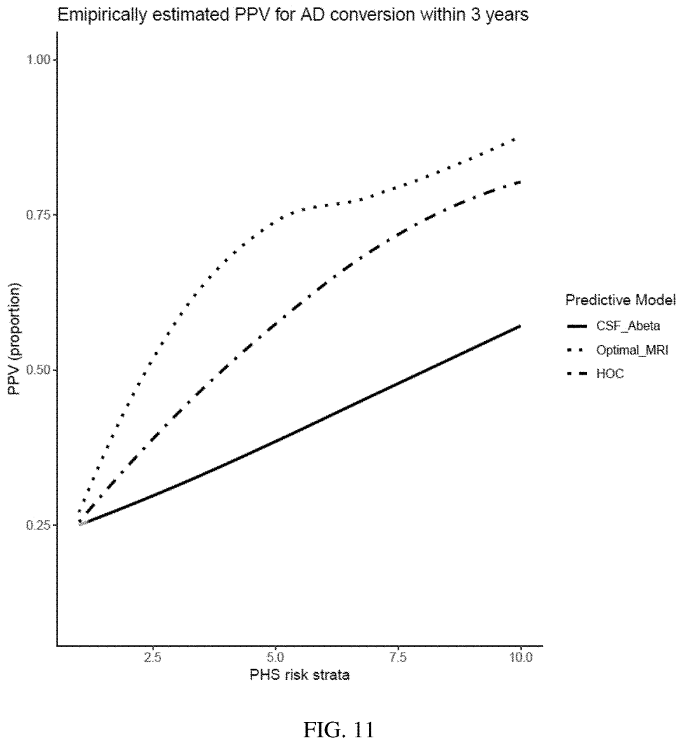

Using the same ADNI cohort, we demonstrated the utility of our combined approach. First, we established an optimal threshold for differentiating cases and controls in the ADNI cross-sectional data for each biomarker (HOC, Optimal MRI, and CSF-Abeta). We then applied the given test and corresponding threshold to determine how well the approach could predict the eventual outcome of an individual in the longitudinal cohort. FIG. 11 illustrates the resulting positive predictive value or PPV. As shown in FIG. 11, The PPV is highest for optimal MRI among those who have high genetic risks. Therefore, compared to other measures, MRI together with genetic risk prediction is the best tool for screening Alzheimer's disease in general population.

Operational embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD- ROM, a DVD disk, or any other form of storage medium known in the art. An exemplary storage medium is coupled to the processor such the processor may read information from, and write information to, the storage medium. In the alternative, the storage medium may be integral to the processor. The processor and the storage medium may reside in an ASIC or may reside as discrete components in another device.

Furthermore, the one or more versions may be implemented as a method, apparatus, or article of manufacture using standard programming and/or engineering techniques to produce software, firmware, hardware, or any combination thereof to control a computer to implement the disclosed embodiments. Non-transitory computer readable media may include but are not limited to magnetic storage devices (e.g., hard disk, floppy disk, magnetic strips), optical disks (e.g., compact disk (CD), digital versatile disk (DVD)), smart cards, and flash memory devices (e.g., card, stick). Those skilled in the art will recognize many modifications may be made to this configuration without departing from the scope of the disclosed embodiments.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method for deriving a personalized health assessment for an individual by integrating selected genotypic information with phenotypic measurements associated with the individual, via a computing system, wherein the computing system
(a) a processor operable to control the computing system,
(b) data storage operatively coupled to the processor, wherein data storage is configured to store a plurality of genotypic information, a plurality of phenotypic measurements, and combinations thereof,
(c) an input/output device operatively coupled to the processor, wherein the input/output device is configured to receive a plurality of data for transmission to the processor, wherein the input/output device is configured to transmit a plurality of data generated by the processor,
(d) a genetic risk prediction component operatively connected to the processor and controlled in part by the processor, wherein the genetic risk prediction component is configured to generate a plurality of genetically defined lifetime risks of having a plurality of diseases, and
(e) an integration component operatively coupled to the processor and controlled in part by the processor, wherein the integration component is configured to integrate genotypic information with phenotypic measurements, the method comprising:
obtaining a plurality of trained genetic risk weights associated with at least one selected medical condition of interest and transmitting at least a portion of the trained genetic risk weights to the genetic risk prediction component;
receiving, via the input/output device, a plurality of germline genetic information associated with the individual and transmitting the received germline genetic information to the genetic risk prediction component;
subjecting, via the genetic risk prediction component, at least a portion of the received germline genetic information to a genetic risk prediction function using at least a portion of the plurality of trained genetic risk weights to generate at least one age-dependent genetic risk score for the individual;
receiving, via the input/output device, a plurality of phenotypic measurement data associated with the individual and transmitting the received phenotypic measurement data to the integration component; and
selectively integrating at least a portion of the received phenotypic measurement data into the at least one age-dependent genetic risk score by the integration component to generate a personalized health assessment for the individual.

2. The method of claim 1, wherein the computing system further comprises a training component operatively connected to the processor and controlled in part by the processor, wherein the training component is configured to generate a plurality of trained genetic risk weights, the method further comprising;
receiving, via the input/output device, a plurality of training genetic risk weights associated with the at least one selected medical condition of interest and transmitting at least a portion of the received training genetic risk weights to the training module;
subjecting at least a portion of the training genetic risk weights to at least one training function by the training module to generate trained genetic risk weights; and
transmitting, via the input/output device, at least a portion of the trained genetic risk weights to the genetic risk prediction component for use in generating the at least one age-dependent genetic risk score.

3. The method of claim 2, wherein the training component further comprises a sample training module, wherein the method further comprises:
determining, by the sample training module, at least one sample parameter for creating a sampling of the plurality of training genetic risk weights;
selecting, by the sample training module, a defined number of the plurality of training genetic risk weights to be included in the sampling in accordance with the at least one sample parameters; and
subjecting the sampling of training genetic risk weights to a resampling process, by the sample training module, to generate trained genetic risk weights.

4. The method of claim 2, wherein the training component further comprises a biological information module, wherein the method further comprises:
receiving, by the input/output device, a plurality of biological information associated with the at least one selected medical condition of interest and transmitting the received biological information to the biological information module; and
selectively incorporating at least a portion of the received biological information into a least a portion of the plurality of training genetic risk weights by the biological information module to generate enhanced genetic risk weights.

5. The method of claim 2, wherein the training component further comprises a summary module, wherein the method further comprises subjecting at least a portion of the plurality of training genetic risk weights to at least one summary transform function by the summary module to generate at least one genetic risk score for the individual.

6. The method of claim 1, wherein the plurality of trained genetic risk weights comprises genetic data selected from the group consisting of genomic data, genotyped calls, imputed genetic data, sequence data, structural variations, copy number variations, and combinations thereof.

7. The method of claim 1, wherein the plurality of germline genetic information comprises data selected from the group consisting of genotype data, genotyped calls, imputed genetic data, sequence data, structural variation data, copy number variations, and combinations thereof.

8. The method of claim 1, wherein the plurality of phenotypic measurement data comprises data selected from the group consisting of biomedical record data, or health care record data, bioassay data, medical imaging data, cognitive performance data, neuropsychological test data, behavioral assessment data, blood analysis data, metabolic test data, physiologic data, and combinations thereof.

9. The method of claim 3, wherein the sampling of training genetic risk weights is subjected to a penalized regression process, by the sample training module, to generate trained genetic risk weights.

10. The method of claim 4, wherein the plurality of received biological information comprises data selected from the group consisting of genic positional annotation data, pleiotropic trait data, gene function data, mutation impact data, predicted functional impact data, genome 3D structure data, and combinations thereof.

11. The method of claim 10, further comprising:
receiving, via the input/output device, a plurality of biological information associated with at least one ancillary medical condition and transmitting the received biological information to the biological information module; and
selectively incorporating at least a portion of the received biological information associated with the at least one ancillary medical condition into a least a portion of the plurality of training genetic risk weights by the biological information module to generate enhanced genetic risk weights.

12. The method of claim 5, wherein the summary transform function comprises transform functions selected from the group consisting of linear transform functions, exponential transform functions, polynomial transform functions, and combinations thereof.

13. The method of claim 1, wherein at least a portion of the received phenotypic measurement data is selectively integrated into the at least one age-dependent genetic risk score by the integration component using the Bayes rule.

14. The method of claim 1, further comprising:
receiving, via the input/output device, a plurality of updated phenotypic measurement data associated with the individual and transmitting the updated phenotypic measurement data to the integration component; and
selectively integrating at least a portion of the updated phenotypic measurement data into the at least one age-dependent genetic risk score by the integration component to generate an updated personalized health assessment for the individual.

15. The method of claim 14, further comprising
receiving, via the input/output device, a plurality of genetically informed population normative data associated with at least one medical condition and transmitting the received genetically informed population normative data to the integration component;
selectively integrating at least a portion of the genetically informed population normative data into the at least one age-dependent genetic risk score by the integration component to generate an augmented personalized health assessment for the individual.

16. The method of claim 1, wherein the personalized health assessment for the individual comprises health prediction data selected from the group consisting of predicted age of onset for a selected medical condition, predicted health costs for the individual, cost/benefit analysis data of updating phenotypic measurement data associated with the individual, predicted life expectancy of the individual, and combinations thereof.

17. A system for deriving a personalized health assessment for an individual by integrating selected genotypic information with phenotypic measurements associated with the individual, the system comprising
a processor operable to control the computing system,
data storage operatively coupled to the processor, wherein data storage is configured to store a plurality of genotypic information, a plurality of phenotypic measurements, and combinations thereof,
an input/output device operatively coupled to the processor, wherein the input/output device is configured to receive a plurality of data for transmission to the processor, wherein the input/output device is configured to transmit a plurality of data generated by the processor, wherein the input/output device is configured to receive a plurality of trained genetic risk weights associated with a selected medical condition, a plurality of germline genetic information associated with the individual, and a plurality of phenotypic measurement data associated with the individual;
a genetic risk prediction component operatively connected to the processor and controlled in part by the processor, wherein the genetic risk prediction component is configured to generate a plurality of genetically defined lifetime risks of having a plurality of diseases, and
an integration component operatively coupled to the processor and controlled in part by the processor, wherein the integration component is configured to integrate genotypic information with phenotypic measurements;
wherein the input/output device is operable to:
receive a plurality of trained genetic risk weights associated with at least one selected medical condition and transmit at least a portion of the trained genetic risk weights to the genetic risk prediction component,
receive a plurality of germline genetic information associated with the individual and transmit the received germline genetic information to the genetic risk prediction module, and
receive a plurality of phenotypic measurement data associated with the individual and transmit the received phenotypic measurement data to the integration component;
wherein the genetic risk prediction component is operable to:
receive at least a portion of the trained genetic risk weights from the input/output device, and
receive at least a portion of the germline genetic information from the input/output device and subject at least a portion of the received germline genetic information to a genetic risk prediction function using at least a portion of the trained genetic risk weights to generate at least one age-dependent genetic risk score for the individual;
wherein the integration component is operable to:
receive at least a portion of phenotypic measurement data associated with the individual, and
selectively integrate at least a portion of the received phenotypic measurement data into the at least one age-dependent genetic risk score to generate a personalized health assessment for the individual.

18. The system of claim 17, wherein the genetic risk prediction component further comprises a training component operatively connected to the processor and controlled in part by the processor, wherein the training component is configured to generate a plurality of trained genetic risk weights,
wherein the input/output device is further operable to:
receive a plurality of training genetic risk weights associated with the at least one selected medical condition and transmit at least a portion of the plurality of training genetic risk weights to the training component, and
transmit at least a portion of the trained genetic risk weights to the genetic risk prediction component for use in generating the at least one age-dependent genetic risk score;
wherein the training component is operable to:
receive at least portion of the plurality of training genetic risk weights from the input/output device,
subject at least a portion of the plurality of training genetic risk weights to at least one training function to generate trained genetic risk weights, and
transmit at least a portion of the trained genetic risk weights to the input/output device.

19. A method for deriving a genetic risk score for an individual via a computing system, wherein the computing system
(a) a processor operable to control the computing system,
(b) data storage operatively coupled to the processor, wherein data storage is configured to store a plurality of genotypic information,
(c) an input/output device operatively coupled to the processor, wherein the input/output device is configured to receive a plurality of data for transmission to the processor, wherein the input/output device is configured to transmit a plurality of data generated by the processor,
(d) a genetic risk prediction component operatively connected to the processor and controlled in part by the processor, wherein the genetic risk prediction component is configured to generate a plurality of genetically defined lifetime risks of having a plurality of diseases, and
obtaining a plurality of trained genetic risk weights associated with at least one selected medical condition and transmitting at least a portion of the trained genetic risk weights to the genetic risk prediction component;
receiving, via the input/output device, a plurality of germline genetic information associated with the individual and transmitting the received germline genetic information to the genetic risk prediction module; and
subjecting, via the genetic risk prediction component, at least a portion of the received germline genetic information to a genetic risk prediction function using at least a portion of the plurality of trained genetic risk weights to generate at least one age-dependent genetic risk score for the individual.

20. The method of claim 19, wherein the computing system further comprises an integration component operatively coupled to the processor and controlled in part by the processor, wherein the integration component is configured to integrate genotypic information with phenotypic measurements, wherein the method further comprises
receiving, via the input/output device, a plurality of phenotypic measurement data associated with the individual and transmitting the received phenotypic measurement data to the integration component; and
selectively integrating at least a portion of the received phenotypic measurement data into the at least one age-dependent genetic risk score by the integration component to generate a personalized health assessment for the individual.
